(19) United States
(12) Patent Application Publication
(10) Pub. No.: US 2025/0228492 A1
(43) Pub. Date: Jul. 17, 2025

(54) SYSTEMS, METHODS, AND APPARATUSES FOR FETAL MONITORING

(71) Applicant: United States Government as Represented by the Department of Veterans Affairs, Washington, DC (US)

(72) Inventors: Curtis Lowery, Honolulu, HI (US); Hari Eswaran, Little Rock, AR (US); James D. Wilson, Benton, AR (US)

(21) Appl. No.: 19/016,679

(22) Filed: Jan. 10, 2025

Related U.S. Application Data
(60) Provisional application No. 63/619,902, filed on Jan. 11, 2024.

(51) Int. Cl. A61B 5/00

(52) U.S. Cl. CPC A61B 5/4356; A61B 5/6823

(57) ABSTRACT

Methods, systems, and apparatuses are described for a remote fetal sensor device. For example, the remote fetal sensor device may detect a first signal measurement from a first sensor device and a second signal measurement from a second sensor device. For example, the first signal measurement may be indicative of a first parameter. The second signal measurement may be indicative of a second parameter. Based on at least one of the first parameter or the second parameter, a contractile event indicative of a uterine contraction for a pregnant person may be determined.

### CROSS-REFERENCE TO RELATED APPLICATION

This application claims priority to U.S. Provisional Application No. 63/619,902, filed on Jan. 11, 2024, the entirety of which is incorporated by reference herein.

### BACKGROUND

A tocodynamometer, known as a TOCO or uterine contraction monitor, is a medical device used to measure and record uterine contractions during pregnancy and labor for a person. The tocodynamometer is an important tool to monitor the progress of labor and assess the frequency, duration, and intensity of contractions for the person. The tocodynamometer helps healthcare providers monitor the frequency and strength of contractions and assess whether the labor is progressing normally. However, the accuracy of a tocodynamometer depends on various factors, such as an external placement on the abdomen, the quality of the device, and uterine contraction variation from person to person. For example, if the tocodynamometer is not appropriately positioned or if there is a lot of movement, the measurement is less accurate. About 20% of the time, the device produces less accurate results due to various factors.

### SUMMARY

It is understood that both the following general description and the following detailed description are example and explanatory only and are not restrictive.

Methods, systems, and apparatuses are described for remote fetal monitoring. For example, a computing device may receive, from a first sensor device, a first signal measurement. The first sensor device may comprise at least one electrode. The first signal measurement may comprise one or more electrical signals. The first signal measurement may indicate a first parameter. The computing device may receive, from a second sensor device, a second signal measurement. The second sensor device may be a pneumatic tocodynamometer (pTOCO). The second signal measurement may comprise one or more pressure changes measured at the second sensor device. The second signal measurement may indicate a second parameter. The computing device may determine, based on at least one of the first parameter or the second parameter, a contractile event indicative of a uterine contraction.

Additional advantages will be set forth in part in the description which follows or may be learned by practice. The advantages will be realized and attained by means of the elements and combinations particularly pointed out in the appended claims. It is to be understood that both the foregoing general description and the following detailed description are example and explanatory only and are not restrictive.

### BRIEF DESCRIPTION OF THE DRAWINGS

The accompanying drawings, which are incorporated in and constitute a part of the present description, serve to explain the principles of the methods and systems described herein. To easily identify the discussion of any particular element or act, the most significant digit or digits in a reference number may refer to the figure number in which that element is first introduced.

FIG. 1 shows an example communication system;

FIG. 2A is a rear perspective view of an example device;

FIG. 2B is a side perspective view of the example device;

FIG. 2C is a rear perspective view of the example device;

FIG. 2D is a bottom perspective view of the example device;

FIG. 2E is a front perspective view of the example device;

FIG. 2F is a top perspective view of the example device;

FIG. 3 shows an example device;

FIG. 4 shows an example method;

FIG. 5 shows an example method; and

FIG. 6 shows an example computing device.

### DETAILED DESCRIPTION

Before the present methods and systems are disclosed and described, it is to be understood that the methods, apparatuses, and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed, that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods, apparatuses, and systems may be understood more readily by reference to the following detailed description of example embodiments and to the figures and their previous and following description.

As will be appreciated by one skilled in the art, that the methods, apparatuses, and systems may take the form of an entirely hardware embodiment, an entirely software embodiment, or an embodiment combining software and hardware aspects. Furthermore, a computer program product on a computer-readable storage medium (e.g., non-transitory) having processor-executable instructions (e.g., computer software) embodied in the storage medium. More particularly, the present methods and systems may take the form of web-implemented computer software. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Example embodiments of the methods, apparatuses, and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These processor-executable instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These processor-executable instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Hereinafter, various embodiments of the present disclosure will be described with reference to the accompanying drawings. As used herein, the terms “user,” or “subject,” may indicate a person who uses an electronic device or a device (e.g., an artificial intelligence electronic device) that uses an electronic device.

The methods, apparatuses, and systems described herein may allow for assessment of contractile events in inpatient or outpatient settings and is not limited to use only in obstetrics. For example, a device or system that implements the methods described herein may be intended as a portable, point of care instrument that can be used in inpatient and outpatient settings and can be used with little formal training by both providers and patients in the home environment.

The methods, apparatuses, and systems described herein may provide a low cost alternative for fetal monitoring that is flexible enough to fulfill the needs of labor and delivery, emergency room, doctor's office, home testing, and telemedicine applications. The device and/or system implementing the methods may comprise minimal hardware such as a set of electrodes (e.g., five electrodes), a pneumatic tocodynamometer (pTOCO), a 32-bit microcontroller (e.g., a multicore processor), and a wireless transmitter/receiver unit. The electrodes and pTOCO may be disposable. In addition, a web-based analysis and human interface application may be used with the device and/or system. For example, the web-based application and the methods or processes described herein may run or be implemented on a personal computer, tablet, smart phone, smart watch, a cell phone or the like.

The methods, apparatuses, and systems described herein provide various improvements to assessing contractile events. A tocodynamometer, known as a TOCO or uterine contraction monitor, is a medical device used to measure and record uterine contractions during pregnancy and labor for a person. However, tocodynamometers may often be less accurate due to various factors. For instance, if the tocodynamometers are improperly placed on a patient's abdomen, the accuracy of identifying contractile events significantly decreases. Because each patient could have unique anatomy, identifying the appropriate placement can be significant to ensuring the accuracy. About 20% of the time, tocodynamometers produce less accurate results than what is medically required. To address these problems, the methods, apparatuses, and systems described herein provide one or more sensor devices, such as electrodes and pneumatic tocodynamometers (pTOCO). The sensor devices may identify changes in muscular tension in the uterus that is brought about by the electrical depolarization of the smooth muscles in the uterus. However, maternal movement can create a false pressure reading in a pTOCO, so concurrent observation of an electrohysterogram (EHG), captured by the same electrodes or pTOCO, can validate whether a contraction is occurring.

Additionally, the various methods, apparatuses, and systems described herein provide various improvements to communications between computing devices. At least one problem in computing is that it can be challenging to transmit large amounts of data over a network. In the medical setting, being able to communicate information to various devices that may have limited or specialized computing resources over a network can be especially challenging. For example, the data acquisition or monitoring systems can stream all the data in real time to other units or other devices (e.g., a physician's device, a monitor, etc.). Large data sets often consume significant bandwidth when transferred over a network. In ‘at home’ situations (e.g., telemedicine cases), patients may have even more significantly limited bandwidth. For example, a patient may have limited bandwidth at their home. In another example, a patient may have limited bandwidth because their devices are communicating via a cellular network. In such situations, the bandwidth required to transmit high resolution fetal ECG may not be sufficient. Thus, methods, apparatuses, and/or systems that are able to adapt to provide needed data in a low bandwidth environment are beneficial, especially, when the patient is located in a low bandwidth wireless/wired environment. For example, a web application may request a fixed duration recording. Upon analysis, the web application may transmit back the coefficients of a data driven beamformer. Such a beamformer may be implemented by a 32-bit processor in real time. The purpose of the beamformer may be to preprocess the raw recording to suppress all signals except the fetal ECG ‘R’ waves, thus allowing the data recorder to efficiently extract the fetal beat to beat interval (e.g., heart rate). By isolating the specific fetal ECG ‘R’ waves, such computing devices may reduce the needed bandwidth dramatically. The telemedicine providers may observe the uterine activity and fetal heart rate in real time, and may review short recordings of the raw abdominal recordings. Additionally, the methods/apparatuses/systems described herein may also be able to give an indication to the mother of fetal heartbeat via a blinking LED.

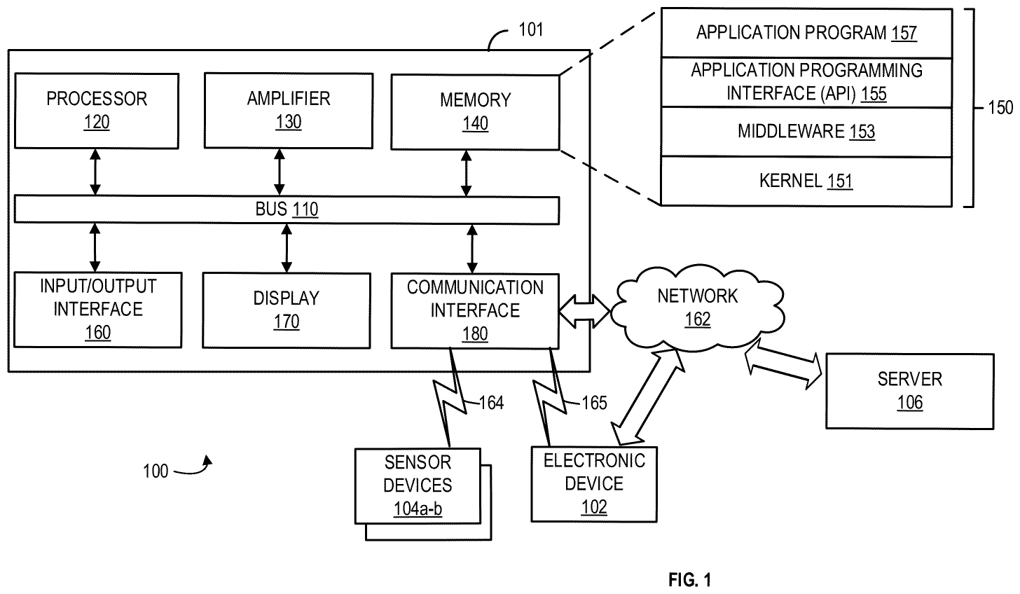

FIG. 1 shows an example communication system 100, where the methods, apparatuses, and systems described herein may be implemented according to various embodiments. Referring to FIG. 1, a computing device 101 in the communication system 100 is disclosed according to various example embodiments. The computing device 101 may include a bus 110, a processor 120, an amplifier 130, a memory 140, an input/output interface 160, a display 170, and a communication interface 180. In a certain example embodiment, the computing device 101 may omit at least one of the aforementioned elements or may additionally include other elements. The computing device 101 may comprise a microcomputer, a miniature computer, a single board computer, a microcontroller, or a circuit board. For example, the computing device 101 may be a mobile phone, a smart phone, a tablet computer, a laptop computer, a desktop computer, a smartwatch, and the like.

The computing device 101 may be configured to process the signals or data received from other devices such as the one or more sensor devices or systems 104a-b. The computing device 101 may receive the signals or data using a wireless connection or a wired connection. For example, the computing device 101 may forward the received signals or data to an external electronic device 102 and/or a server 106 for further processing. The external electronic device 102 and/or the server 106 may process the signals or data and transmit the processed data to the computing device 101.

The bus 110 may include a circuit for connecting the aforementioned elements 110 to 180 to each other and for delivering communication (e.g., a control message and/or data) between the aforementioned elements 110 to 180. For instance, the bus 110 may be designed to send the signals or sensor data from the processor 120 to the communication interface 180 in order to further transmit the signals or sensor data to an external device, such as the electronic device 102 and/or a server 106.

The processor 120 may include one or more of a Microcontroller Unit (MCU), a Central Processing Unit (CPU), an Application Processor (AP), or a Communication Processor (CP). The processor 120 may control, for example, at least one of the other elements of the computing device 101 and/or may execute arithmetic operations or data processing for communication. The processing (or controlling) operation of the processor 120, according to various examples, is described in detail with reference to the following figures. The processor 120 may include an on-chip analog-to-digital converter (ADC) for converting the amplified voltage signal, received from the amplifier 130 (described below), from an analog signal to a digital signal. The processor 120 may be used to process the digital signal in order to calculate the waveform/force applied to each sensor location in Newtons, and the associated pressure applied to the sensor devices 104a-b in mmHg. The processor 120 may then send the processed data, including the signal, waveform, force, and/or pressure data, to the communication interface 180 (e.g., a Bluetooth module, a cellular module, a Wi-Fi module, a Zigbee module, an NFC module, or any other short/long range communication module) using a Universal Asynchronous Receiver/Transmitter (UART), wherein the communication interface 180 may further transmit the signal, waveform, force and/or pressure data to an external electronic device 102, such as a smart phone, or a server 106.

The processor 120 may receive, from a first sensor device or system 104a, a first signal measurement. The first sensor device or system 104a may comprise at least one electrode. In certain examples, the first sensor device comprises four or five electrodes. The first sensor device or system 104a may detect one or more first signal measurements. The first signal measurement may comprise one or more electrical signals. The first signal measurement may indicate a first parameter. For example, the first signal measurement may indicate a heartrate of the fetus (e.g., fetal heartrate). The processor 120 may receive, from a second sensor device or system 104b, a second signal measurement. The second sensor device or system 104b may be a pneumatic tocodynamometer (pTOCO). The second signal measurement may indicate a second parameter. For example, the second parameter may comprise or indicate one or more pressure changes along the abdomen of the pregnant person measured at the second sensor device or system 104b. The processor 120 may determine, based on at least one of the first parameter or the second parameter, a contractile event indicative of a uterine contraction occurring within the pregnant person. For example, if the first parameter satisfies a first threshold but the second parameter does not satisfy a second threshold, the processor 120 may determine that the uterine contraction has occurred within the pregnant person (e.g., a positive uterine contraction). In another example, if the first parameter does not satisfy a first threshold but the second parameter satisfies a second threshold, the processor 120 may determine the uterine contraction has occurred within the pregnant person (e.g., a positive uterine contraction). In another example, if the first parameter satisfies a first threshold and the second parameter satisfies a second threshold, the processor 120 may determine that the uterine contraction has occurred within the pregnant person (e.g., a positive uterine contraction). For example, if neither the first parameter satisfies a first threshold nor the second parameter satisfies a second threshold, the processor 120 may determine that the uterine contraction has not occurred within the pregnant person (e.g., a negative uterine contraction). Once the contractile event is determined to have occurred within the pregnant person, the result of the contractile event may be sent to other devices such as an electronic device 102 and/or a server 106 via a communication interface 180.

The amplifier 130 may include an instrumentation amplifier, such as a MAX4208. An amplifier 130 may be used for the first sensor device 104a in order to amplify the signal or signals received from the first sensor device 104a. The amplifier 130 may be used for the second sensor device 104b in order to amplify the signal or signals received from the second sensor device 104b. The signals from the first sensor device 104a and/or the second sensor device 104b may be very small relative to the supply voltage. The output signal may be amplified using the amplifier 130 in order to obtain optimal results from the ADC.

The memory 140 may include volatile and/or non-volatile memory. The memory 140 may store, for example, a command or data related to at least one different element of the computing device 101. According to various example embodiments, the memory 140 may store a software and/or a program 150. The program 150 may include, for example, a kernel 151, a middleware 153, an Application Programming Interface (API) 155, and/or an application program (or an “application”) 157, or the like, configured for controlling one or more functions of the computing device 101 and/or an external device. At least one part of the kernel 151, middleware 153, or API 155 may be referred to as an Operating System (OS). The memory 140 may include a computer-readable recording medium having a program recorded therein to perform the method according to various embodiments by the processor 120.

The kernel 151 may control or manage, for example, system resources (e.g., the bus 110, the processor 120, the memory 130, etc.) used to execute an operation or function implemented in other programs (e.g., the middleware 153, the API 155, or the application program 157). Further, the kernel 151 may provide an interface capable of controlling or managing the system resources by accessing individual elements of the computing device 101 in the middleware 153, the API 155, or the application program 157.

The middleware 153 may perform, for example, a mediation role so that the API 155 or the application program 157 can communicate with the kernel 151 to exchange data.

Further, the middleware 153 may handle one or more task requests received from the application program 157 according to a priority. For example, the middleware 153 may assign a priority of using the system resources (e.g., the bus 110, the processor 120, or the memory 140) of the computing device 101 to at least one of the application programs 157. For instance, the middleware 153 may process the one or more task requests according to the priority assigned to at least one of the application programs, and thus, may perform scheduling or load balancing on the one or more task requests.

The API 155 may include at least one interface or function (e.g., an instruction), for example, for file control, window control, video processing, or character control, as an interface capable of controlling a function provided by the application program 157 in the kernel 151 or the middleware 153.

For example, the input/output interface 160 may play a role of an interface for delivering an instruction or data input from a user or a different external device(s) to the different elements of the computing device 101. Further, the input/output interface 160 may output an instruction or data received from the different element(s) of the computing device 101 to the different external device.

The display 170 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. The display 170 may display, for example, a variety of contents (e.g., text, image, video, icon, symbol, etc.) to the user. The display 170 may include a touch screen. For example, the display 170 may receive a touch, gesture, proximity, or hovering input by using a stylus pen or a part of a user's body.

The communication interface 180 may establish, for example, communication between the computing device 101 and an external device (e.g., an electronic device 102, sensor devices 104a-b, or a server 106). In one example, the communication interface 180 may communicate with the first sensor device 104a and/or the second sensor device 104b (e.g., the pTOCO) through wireless communication or wired communication. In one example, the communication interface 180 may communicate with the external device (e.g., the electronic device 102 and/or the server 106) by being connected to a network 162 through wireless communication or wired communication.

In another example, as a cellular communication protocol, the wireless communication may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. Further, the wireless communication may include, for example, a near-distance communication 164, 165. The near-distance communications 164, 165 may include, for example, at least one of Bluetooth, Wireless Fidelity (WiFi), Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. The wired communication interface may include, for example, at least one of Universal Serial Bus (USB), High Definition Multimedia Interface (HDMI), Recommended Standard-232 (RS-232), power-line communication, Plain Old Telephone Service (POTS), and the like. The network 162 may include, for example, at least one of a telecommunications network, a computer network (e.g., LAN or WAN), the internet, and a telephone network.

For example, the computing device 101 may forward the signals or data, received from the sensor devices 104a-b, to a server 106 for processing via the network 162. The server 106 may then transmit the processed data to the electronic device 102 via the network 162. In another embodiment, the computing device 101 may forward the signals or data, received from the sensor devices 104a-b, to the electronic device 102 to further process the received signals or data.

The electronic device 102 may comprise a mobile phone, a smart phone, a tablet computer, a laptop computer, a desktop computer, a smartwatch, and the like. The electronic device 102 may receive sensing data of the first sensor device 104a from the computing device 101 via the communication interface 180. The electronic device 102 may also receive sensing data of the second sensor device 104b from the computing device 101 via the communication interface 180. The electronic device 102 may then output the received signal data and/or sensor data to the user. For example, the electronic device 102 may receive the signal data from the server 106 via network 162. The electronic device 102 may receive the sensor data from the server 106 via network 162. For example, the server 106 may receive the signal and/or sensor data from the computing device 101 and perform further processing. The server 106 may then transmit the signal/sensor data or the processed signal/sensor data to the electronic device 102 to be output to the user (e.g., a physician, nurse, medical technician, etc.). For example, the computing device 101 may transmit the signal data or sensor data to the electronic device 102 for further processing. For example, the electronic device 102 may process the signal data or sensor data in order to cross-validate based on the received signal data and the received sensor data and/or adaptive bandwidth adjustment. For example, the electronic device 102 may include a smartphone application for interfacing with the sensor data and displaying the sensor data to the user.

Each of the sensor devices 104a-b may comprise all the components necessary to capture, process, and store electrical signal information. For example, the first sensor device 104a may comprise one or more electrodes placed on the abdomen of a pregnant person to detect the plurality of electrical signals associated with a fetal heartrate from a fetal heart or a uterine contraction. The one or more electrodes may comprise electrical conductors that are used to make contact with a non-metallic part of a circuit. The one or more electrodes may be used for electrocardiograms (ECGs or EKGs) to measure and record the electrical activity of the heart of the fetus or a uterine contraction. Electrode materials may include metals like copper, platinum, and graphite, as well as conductive polymers and certain types of metal oxides.

The first sensor device 104a may have one or more built-in transducers or may include one or more separate transducers. For example, one or more built-in transducers or one or more separate transducers may be operatively coupled to the one or more electrodes, to convert one form of energy to another. The transducer connected to or integrated into the first sensor device 104a may allow the conversion of a physical parameter or signal into an electrical signal, which can then be easily measured, processed, and analyzed. The electrical signal can be in the form of voltage, current, or resistance, depending on the type of transducer and the specific application.

The second sensor device 104b may be a tocodynamometer, also known as a TOCO or uterine contraction monitor. The second sensor device 104b may be used to measure and record uterine contractions during pregnancy and labor. It may be used to monitor the progress of labor for a pregnant person and assess the frequency, duration, and intensity of contractions in the pregnant person. The second sensor device 104b may have one or more built-in transducers or may include one or more separate transducers. For example, the second sensor device 104b may comprise a tocotransducer and/or a monitoring unit. The tocotransducer may be a pressure-sensitive device that is placed on the abdomen of the pregnant person, usually over the fundus (e.g., the upper part) of the uterus. The tocotransducer may detect changes in uterine pressure caused by contractions. The monitoring unit may receive the signals from the tocotransducer and display the uterine contractions graphically on a monitor or record them on a paper strip. The graphical representation may show the intensity and duration of contractions over time, allowing healthcare providers to assess the progress of labor.

For example, the second sensor device 104b may be a pneumatic tocodynamometer, or pTOCO. The second sensor device 104b may use a trapped air volume surrounded by a “guard ring” arrangement. The pTOCO may be equivalent in performance to “guard ring” tocodynamometers. The pTOCO may be a very low cost tocodynamometer and may allow for the pTOCO to be a disposable device that can be provided to a pregnant person for a single term use. The pTOCO may provide useful information about the fetal or maternal heart rate or contraction of the pregnant person with additional devices. For example, with a low-end microcontroller, the pTOCO may capture uterine contractions during pregnancy and labor. Captured data may be sent, via a wireless transmitter/receiver module, to the computing device 101, the electronic device 102, and/or the server 106. For example, the wireless transmitter/receiver may be communicably coupled to the first sensor device or system 104a and the second sensor device or system 104b.

For example, the server 106 may include a group of one or more servers. According to various example embodiments, all or some of the operations executed by the computing device 101 may be executed in a different one or a plurality of electronic devices (e.g., the electronic device 102 or the server 106). For example, the processing of the data received from the sensor devices 104a-b may be performed by the electronic device 102 and/or the server 106. According to one example embodiment, if the computing device 101 needs to perform a certain function or service either automatically or based on a request, the computing device 101 may request at least some parts of functions related thereto alternatively or additionally to a different electronic device (e.g., the electronic device 102 or the server 106) instead of executing the function or the service autonomously. The different electronic devices (e.g., the electronic device 102 or the server 106) may execute the requested function or additional function, and may deliver a result thereof to the computing device 101 or to the electronic device 102. The computing device 101 may provide the requested function or service either directly or by additionally processing the received result. For this, for example, a cloud computing, distributed computing, or client-server computing technique may be used.

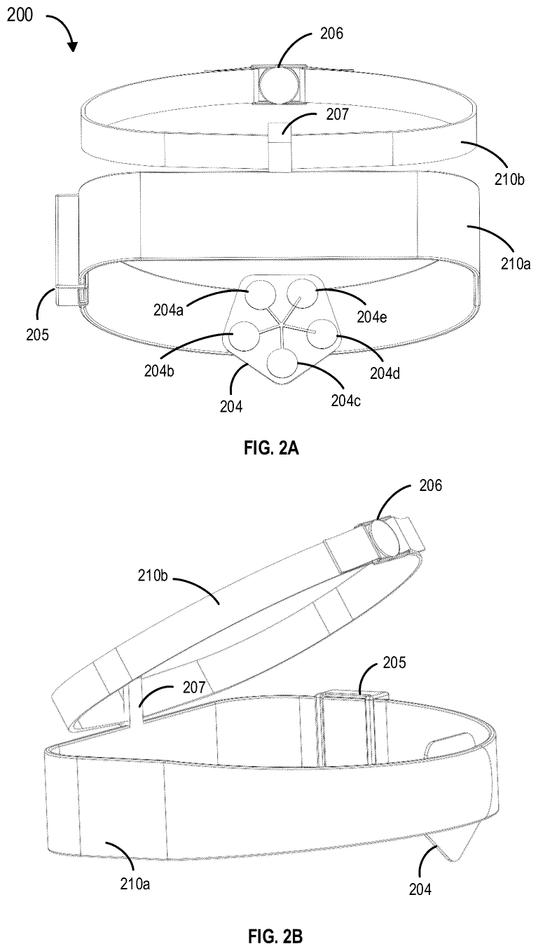

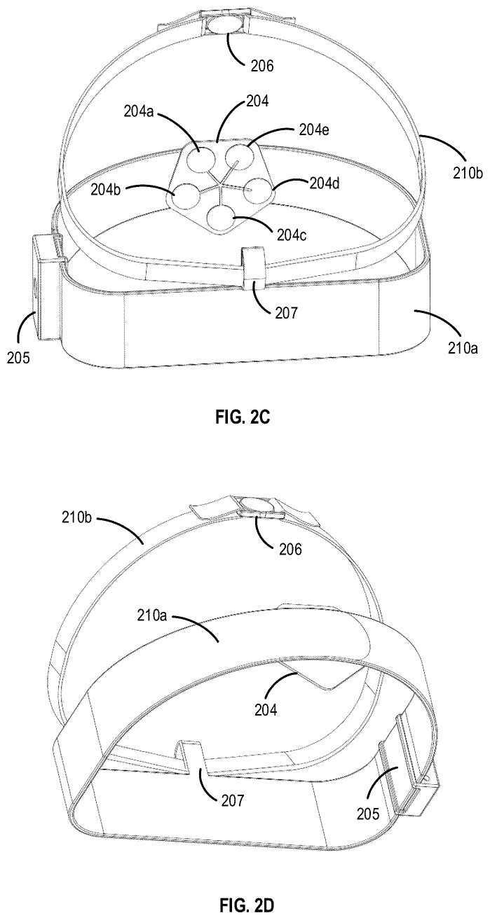

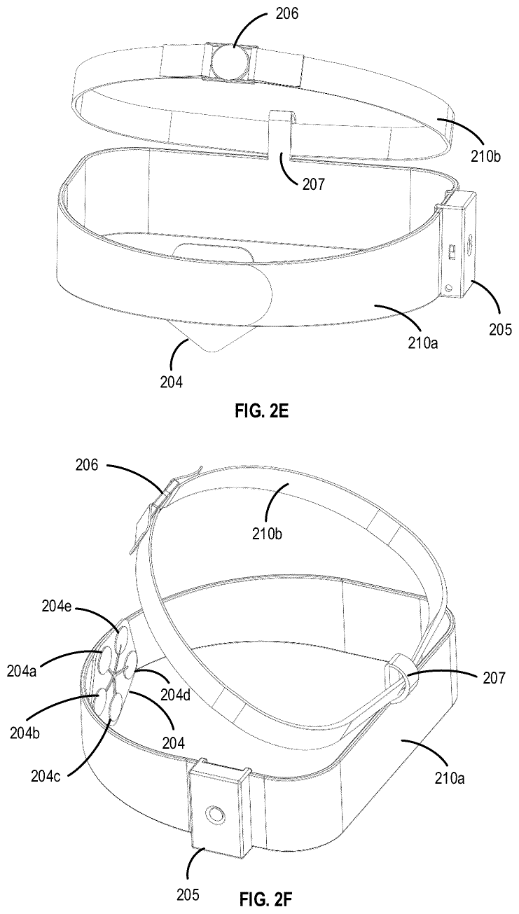

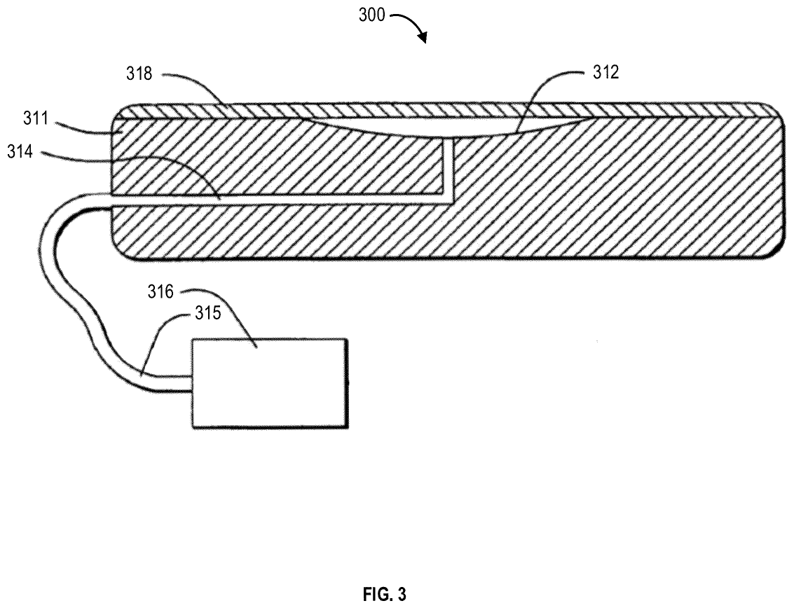

FIG. 2A shows an example device 200 for monitoring/detecting one or more uterine contractions in a pregnant person. FIGS. 2B-2F are example devices 200 from different aspects, which may be used in combination with any of other embodiments described herein. The example device 200 may be used in combination with any of other elements described herein. As shown in FIG. 2A, the device 200 may comprise one or more belts 210a-b (e.g., a first belt or first belt portion 210a and a second belt or second belt portion 210b), a sensor system (e.g., a first sensor device or system 204 and a second sensor device or system 206), and a computing device 205. The first sensor device or system 204 may comprise a plurality of sensors. For example, the first sensor device or system may comprise a plate (or a flexible pad) and one or more electrodes 204a-e. The one or more electrodes 204a-e may be disposed on the surface of the plate or the flexible pad. The one or more electrodes 204a-e may extend out from the plate or the flexible pad, for example, by one or more wires to allow for more precise placement. The first sensor device or system 204 may be coupled to the belt 210a (e.g., first belt or first belt portion) and the computing device 205. For example, the first sensor device or system 204 may be coupled to a first portion of the belt 210a. The second sensor device or system 206 may be coupled to the belt 210b (e.g., the second belt or second belt portion) and the computing device 205. For example, the second sensor device or system 206 may be coupled to a portion (e.g., a first portion) of the belt 210b. For example, the second sensor device 206 may be disposed on the surface of the belt 210b. For example, the second sensor device 206 may extend out from the surface of the belt 210b for more precise placement. The belt or belt portion 210a and the belt or belt portion 210b may be coupled to each other using a hinge coupling or mechanism 207. The hinge coupling or mechanism 207 may hold the two belts 210a-b and allow for rotation of one belt with respect to the other along two axes. For example, the second belt or second belt portion 210b may be configured to rotate about a first axis with respect to the first belt or first belt portion 210a by way of the hinge coupling 207. The hinge mechanism may be any component that allows flexibility and movement at the connection point of the two belts 210a-b.

As described above, the first sensor device 204 may comprise the plate (or the flexible pad) and the one or more electrodes 204a-e. The one or more electrodes 204a-e may be placed on an abdomen of a pregnant person to detect a plurality of electrical signals associated with a fetal heartrate of the fetus and/or uterine contractions. The first sensor device 204 may be used in electrocardiograms (ECG (Electrocardiography) and EHG (Electrogastrography)) to measure and record the electrical activity of the fetal heartrate and/or uterine contraction.

For example, one or more electrical signals may be detected and measured by the one or more electrodes 204a-e. For example, in the context of ECG (Electrocardiogram) and EHG (Electrogastrography), the one or more electrodes 204a-e may be used to measure different types of electrical signals. In the ECG, the one or more electrodes 204a-e may measure electrical impulses generated by uterine depolarization and repolarization as it contracts and relaxes. In an example, the one or more electrodes 204a-e may be placed on the skin of the pregnant person at specific locations on the body, such as the chest, arms, abdomen and legs, to detect these electrical signals. In the EHG, the one or more electrodes 204a-e may be used to measure the electrical activity of fetal heartrate and/or muscle contraction. This technique may help monitor the slow waves produced by the smooth muscles where the one or more electrodes 204a-e are placed. The one or more electrodes 204a-e may be placed on the abdominal surface to record these electrical signals. The one or more electrodes 204a-e may detect and measure electrical potentials generated by the specific physiological activity of the uterine area of the pregnant person and/or heart function of the fetus. The voltage changes recorded by the one or more electrodes 204a-e may provide insights into when contractions are occurring in the pregnant person.

The first sensor device 204 may be used in electrohysterography (EHG) to measure and record the electrical activity of the uterine muscles. EHG may involve recording the electrical activity of the uterine muscles as they contract during different stages of pregnancy and labor. The procedure may be non-invasive and use the one or more electrodes 204a-e placed on the abdomen of the pregnant person to detect the electrical signals produced by the uterine muscles. These signals are then amplified, for example by the amplifier 130, and recorded to create a graph or waveform representation.

Although it is not shown in FIG. 2A, the first sensor device 204 may have one or more built-in transducers and/or one or more separate transducers communicatively coupled to the one or more electrodes 204a-e, to convert physical characteristics of fetal heartrate and/or uterine contractions (e.g., frequency, intensity, etc.) into one or more electrical signals that can be measured, processed, and used for monitoring, control, or data analysis. For example, the transducer associated with the one or more electrodes 204a-e may convert physiological signals, such as bioelectric potentials (e.g., electrocardiogram, and/or electroencephalogram) or other electrical signals from the body, into measurable electrical signals that can be processed, recorded, or displayed for analysis. For example, the transducer may convert the ionic activity at the electrode-skin interface into one or more electrical signals. These electrical signals may then be amplified and processed to produce a readable output such as waveforms, allowing healthcare providers to evaluate whether a contraction has occurred and other information about that contraction. For example, the transducer for the first sensor device 204 may be embedded in the computing device 205. The computing device 205 may be coupled to the first sensor device 204 and the second sensor device 206 by wires embedded in or disposed outside of the belts 210a-b.

Although it is not shown in FIG. 2A, the first sensor device 204 and/or the transducer associated with the first sensor device 204 may comprise a wired or wireless communication interface to transmit/receive data with other devices. For example, the wireless communication interface associated with the first sensor device 204 may include at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. The wireless communication interface may further include a near-distance communication such as Bluetooth, Wireless Fidelity (WiFi), Near Field Communication (NFC), and Global Navigation Satellite System (GNSS). The wired communication interface may include, for example, at least one of Local Area Network (LAN), Universal Serial Bus (USB), High Definition Multimedia Interface (HDMI), Recommended Standard-232 (RS-232), power-line communication, Plain Old Telephone Service (POTS), and the like.

In an embodiment, the first sensor device 204 may send one or more electrical signals determined by the one or more electrodes 204a-e to the computing device 101, the electronic device 102, and/or a server 106 using wireless connectivity or wired connectivity. For example, a wireless transmitter/receiver may be communicably coupled to the first sensor device or system 204 and the second sensor device or system 204 to facilitate sending electrical signals. The one or more electrical signals may comprise one or more waveforms, which graphically represent one or more signals that show how a physical quantity or variable of fetal heartrate and/or uterine muscles (or pressures) changes over time. The one or more waveforms may be generated by amplifying one or more electrical potentials measured at the one or more electrodes 201a-e based on at least one movement of uterine muscles.

The second sensor device 206 may be a tocodynamometer (TOCO) or a pneumatic tocodynamometer (pTOCO). Although it is not shown in FIG. 2A, the second sensor device 206 may comprise a sensor body and a pressure transducer to detect one or more mechanical signals indicating one or more pressure changes. The one or more pressure changes may be measured by the second sensor device 206 based on at least one movement of uterine muscles. Similar to the first sensor device 204, the second sensor device 206 may send the measured signals (e.g., the one or more mechanical signals measured at the second sensor device 206) to the computing device 205 using wireless communication interfaces or wired communication interfaces. The wireless communication interfaces or wired communication interfaces may be embedded in the second device 206. Although it is not shown in FIG. 2A, the second sensor device 206 may send the measured signals to the computing device 101, the electronic device 102, the sever 106, and/or the like using the wireless communication interfaces or wired communication interfaces. The wireless communication interface associated with the second sensor device 206 may include at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. The wireless communication interface may further include a near-distance communication such as Bluetooth, Wireless Fidelity (WiFi), Near Field Communication (NFC), and Global Navigation Satellite System (GNSS). The wired communication interface may include, for example, at least one of Local Area Network (LAN), Universal Serial Bus (USB), High Definition Multimedia Interface (HDMI), Recommended Standard-232 (RS-232), power-line communication, Plain Old Telephone Service (POTS), and the like.

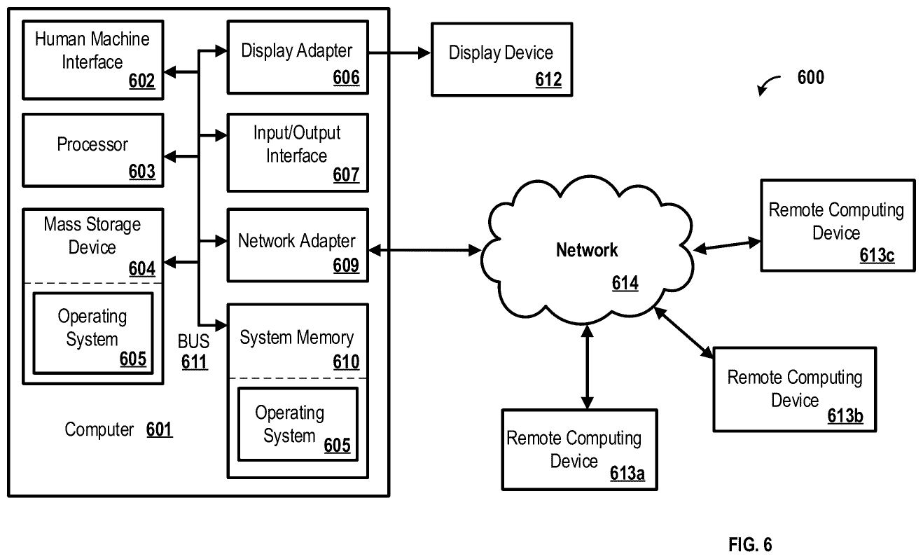

The computing device 205 may be the computing device 101 illustrated in FIG. 1 or a computer 601 illustrated in FIG. 6 or another form of computing device. The computing device 205 may receive, from the first sensor device 204, a first signal measurement. The first signal measurement may comprise the one or more electrical signals measured at the first sensor device 204, as described above. The first signal measurement may indicate a first parameter. The first parameter may comprise one or more metrics, such as values, rhythm, and/or rate indicating electrical activities of the uterus or uterine muscles of the pregnant person. The computing device 205 may receive, from the second sensor device 206, a second signal measurement. The second signal measurement may comprise the one or more pressure changes measured at the second sensor device 206, as described above. The second signal measurement may indicate a second parameter. The second parameter may comprise any metrics, such as values, rhythm, and/or rate indicating pressure changes of the uterus or uterine muscles of the pregnant person. The computing device 205 may determine, based on at least one of the first parameter or the second parameter, whether a contractile event indicative of a uterine contraction has occurred in the pregnant person. In an example, the computing device 205 may compare the first parameter to a first threshold and determine that the first parameter satisfies a first threshold. The computing device 205 may compare the second parameter to a second threshold and determine that the second parameter does not satisfy a second threshold. The computing device 205 may then determine, based on the first parameter satisfying the first threshold and the second parameter not satisfying the second threshold, the contractile event indicative of the uterine contraction occurred in the pregnant person.

In another example, the computing device 205 may determine that the first parameter does not satisfy a first threshold. The computing device 205 may determine that the second parameter satisfies a second threshold. The computing device 205 may determine, based on the first parameter not satisfying the first threshold and based on the second parameter satisfying the second threshold, the contractile event indicative of the uterine contraction occurred in the pregnant person. In another example, the computing device 205 may determine that the first parameter satisfies a first threshold. The computing device 205 may determine that the second parameter satisfies a second threshold. The computing device 205 may then determine, based on the first parameter satisfying the first threshold and based on the second parameter satisfying the second threshold, the contractile event indicative of the uterine contraction occurred in the pregnant person.

The computing device 205 may receive, from the first sensor device 204, a third signal measurement. The third signal measurement may comprise the one or more electrical signals measured at the first sensor device 204, as described above. The third signal measurement may indicate a third parameter, which can be used by the computing device 205 in the same or similar manner as the first parameter, as previously described.

The computing device 205 may receive, from the second sensor device 206, a fourth signal measurement. The fourth signal measurement may comprise the one or more pressure changes measured at the second sensor device 206, as described above. The fourth signal measurement may indicate a fourth parameter, which can be used by the computing device 205 in the same or similar manner as the second parameter, as previously described.

The computing device 205 may send, via a wired or wireless communication interface, at least one of the contractile event indicative of the uterine contraction, the first signal measurement, or the second signal measurement to other devices. The wireless communication interface in the computing device 205 may include at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. The wireless communication interface may include a short-range wireless communication protocol, such as Bluetooth, Wireless Fidelity (WiFi), Near Field Communication (NFC), or Global Navigation Satellite System (GNSS). The wired communication interface may include, for example, at least one of Local Area Network (LAN), Universal Serial Bus (USB), High Definition Multimedia Interface (HDMI), Recommended Standard-232 (RS-232), power-line communication, Plain Old Telephone Service (POTS), and the like.

As described above, monitoring the uterine pressure of the pregnant person with a tocodynamometer may have only an 80% success rate due to various factors such as external placement of the sensors of the tocodynamometer on the abdomen of the pregnant person, the quality of tocodynamometer, and uterine contraction variation from person to person. Based on the data received from the second sensor device 206 (e.g., TOCO or pTOCO data) with the data of the electrohysterogram (EHG), which can be captured concurrently by the one or more electrodes 204a-e of the first sensor device 204 used to record the fetal ECG, may increase the accuracy rate of uterine contraction detection in a pregnant person. The second sensor device 206 (e.g., TOCO or pTOCO) may sense the change in muscular tension in the uterus caused by the electrical depolarization of the smooth muscles in the uterus. In certain situations, maternal movement may create false pressure readings in the second sensor device 206 (e.g., TOCO or pTOCO). However, concurrent observation of the EHG, via the first sensor device 204, may provide a better indication of an actual contraction, however small the contraction might be. Furthermore, EHG signals detected by the one or more electrodes 204a-e may be insufficient to generate viable contractile pressure. That is, EHG alone can give a false positive indication of a contraction. Thus, the second sensor device 206 (e.g., TOCO or pTOCO) may help to validate the sufficiency of the EHG in determining that a contraction has occurred. The EHG may also validate the sufficiently of TOCO or pTOCO in determining that the contraction has occurred.

FIG. 3 shows an example sensor device 300 for monitoring and/or detecting one or more uterine contractions in a pregnant person. The sensor device 300 may be used in combination with any of the other devices described herein. For example, the sensor device 300 may be the second sensor device 206 of FIG. 2A. For example, the sensor device 300 may be a tocodynamometer (TOCO) or a pneumatic tocodynamometer (pTOCO) placed on a abdomen of the pregnant person. As shown in FIG. 3, the sensor device 300 may comprise a sensor body 311. The sensor body 311 may include a shallow concave depression 312. For example, the depression 312 may be located at or near the center of the sensor device 300 and may be surrounded by a guard-ring (not shown in FIG. 3). For example, the shallow depression 312 may be in fluid communication with a low volume airway 314 to a low volume tube 315 fluidically coupled to a pressure transducer 316. In another example, the shallow depression 312 may be directly fluidically coupled to the pressure transducer 316, such as when the pressure transducer 316 is embedded in the sensor body 311. The pressure transducer 316 may be mounted to an electronics board, such as a circuit board. The pressure transducer 316 and electronics board may be positioned within a recess or cavity in the sensor body 311 so that the pressure transducer 316 is in fluid communication with the airway 314 and thence to the air volume in the depression 312. For example, the electronics board may be sealed into the recess or cavity in the sensor body 311. A flexible membrane 318 may be stretched across the sensor body 311 trapping air in a very small volume. The membrane 318 may be attached to the sensor body 311 by adhesive. The sensor body 311 may be made of plastic, such as acetal plastic. The trapped air within the depression 312 plus the air in the airway 314 and air in the tubing 315 may collectively comprise a closed interior air volume or a closed fluid system. For example, pressure applied to the membrane 318 may compress the trapped air, which increases the internal air pressure within the closed fluid system. The pressure applied to the membrane 318 may indicate one or more mechanical signals. The one or more mechanical signals may be converted to one or more waveforms by the pressure transducer 316. The one or more waveforms may be generated by amplifying the pressure changes measured at the membrane 318 based on the movement of uterine muscles of the pregnant person.

In an example, the sensor device 300 may send one or more mechanical signals, measured at the sensor device 300, to the computing device 101, the electronic device 102, and/or a server 106 using wired or wireless connectivity. The one or more mechanical signals may comprise one or more waveforms, which graphically represent one or more signals that show a physical quantity or variable of uterine muscles changes over time. The one or more waveforms may be generated by amplifying one or more pressure values measured at the sensor device 300 based on at least one movement of the uterine muscles of the pregnant person.

Although it is not shown in FIG. 3, the sensor device 300 may comprise a wired or wireless communication interface to transmit/receive data with other devices such as the computing device 101, the electronic device 102, the server 106, and/or the computing device 205. For example, the wireless communication interface in the sensor device 300 may include at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. The wireless communication interface may include short-range communication protocols, such as Bluetooth, Wireless Fidelity (WiFi), Near Field Communication (NFC), and Global Navigation Satellite System (GNSS). The wired communication interface may include, for example, at least one of Local Area Network (LAN), Universal Serial Bus (USB), High Definition Multimedia Interface (HDMI), Recommended Standard-232 (RS-232), power-line communication, Plain Old Telephone Service (POTS), and the like.

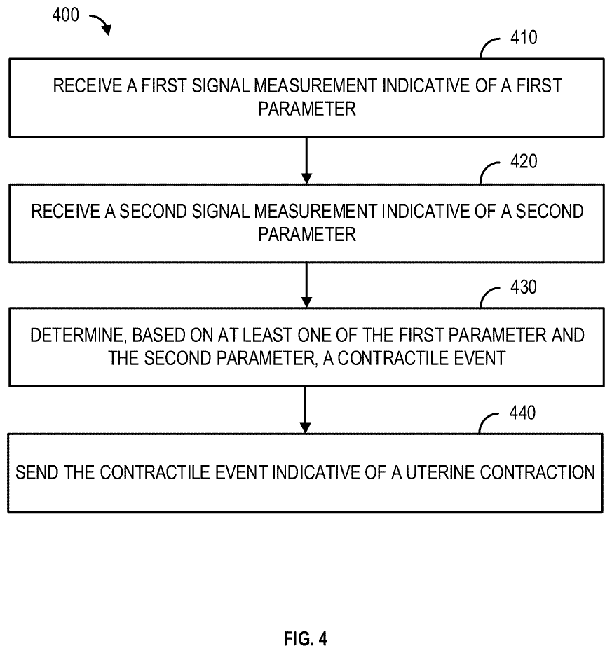

FIG. 4 shows an example method 400 for monitoring and/or detecting one or more uterine contractions in a pregnant person. The method 400 may be used in combination with any of the devices or any of the other embodiments described herein. At 410, a first signal measurement may be received. For example, the computing device 101, 205, 601 may receive the first signal measurement from a first sensor device 104a, 204. The first sensor device 104a, 204 may comprise at least one electrode 204a-e. The first signal measurement may comprise one or more electrical signals. The one or more electrical signals may be measured by the first sensor device 104a, 204. The one or more electrical signals may be produced by or indicative of at least one movement of one or more of the uterine muscles of a pregnant person. The one or more electrical signals may be received using the communication interface 180 of the computing device 101, 601 or wired/wireless connection between the first sensor device 204 and the computing device 205. For example, the first sensor device 104a, 204 may send the one or more electrical signals using a wireless communication interface (e.g., Bluetooth, cellular, Wi-Fi, Zigbee, NFC, or any other short/long range communication interface) equipped with or communicably accessible by the first sensor device 104a, 204. The computing device 101, 205, 601 may receive the one or more electrical signals using the wireless communication interface (e.g., Bluetooth, cellular, Wi-Fi, Zigbee, NFC, or any other short/long range communication interface) equipped with or communicably accessible by the computing device 101, 205, 601. The one or more electrical signals may comprise ECG or EHG signals measured during a period of time. In an example, the one or more electrical signals may comprise one or more waveforms generated by amplifying one or more electrical potentials measured at the first sensor device 104a, 204 based on at least one movement of one or more of the uterine muscles of a pregnant person. The electrical potentials may refer to the voltage differences that exist between two points in a human body. Specifically, the electrical potentials may be generated by the movement of charged particles on the surface of the one or more uterine muscles.

The first signal measurement may indicate a first parameter. The first parameter may be determined based on the one or more electrical signals. For example, the computing device 101, 205, 601 may determine, based on the received one or more electrical signals, the first parameter that is indicative of electrical activity of the heart of a fetus or a uterine contraction. The first parameter may provide valuable information about the amplitude and characteristics of the one or more electrical signals. In case that the one or more electrical signals are EHG signals, the examples of the first parameter may include, but are not limited to, an amplitude (or peak-to-peak voltage), signal-to-noise (SNR), baseline wander, and cross-correlation with the sensor device 104a, 204. Specifically, the amplitude (or peak-to-peak voltage) may measure the difference between the highest positive peak and the lowest negative peak in the EHG signal. A larger amplitude may indicate a stronger signal and a small amplitude may indicate a weak signal. The SNR may represent the ratio of the amplitude of the useful signal (e.g., the uterine contractions) to the amplitude of the background noise. A higher SNR may indicate a stronger and clearer EHG signal. A lower SNR may indicate a weaker and noisy signal. The baseline wander may be the low-frequency fluctuation of the EHG signal baseline caused by various factors, such as movement objects. A lower baseline wander may indicate better signal quality. A higher baseline wander may indicate poorer signal quality. The cross-correlation may measure the correlation between the EHG signal and a reference signal from the sensor device 104a, 204. A higher cross-correlation may indicate stronger agreement between the signals and better signal quality. A lower cross-correlation may indicate weaker agreement between the signals and better signal quality.

In case that the one or more electrical signals are ECG signals, the examples of the first signal measurement may include, but are not limited to, an R-wave amplitude, P-QRS-T complex amplitude, signal quality index (SQI), SNR, and lead voltage levels. The R-wave amplitude may be the tallest peak in the ECG waveform, corresponding to the depolarization of the ventricles. The higher R-wave amplitudes may indicate a stronger ECG signal. The lower R-wave amplitudes may indicate a weaker ECG signal. The P-QRS-T complex amplitude may be the different components of the ECG waveform. Monitoring the amplitude of each complex can provide insights into the strength of atrial and ventricular depolarization and repolarization. The signal quality index (SQI) may be a metric that assesses the overall quality of the ECG signal. It may consider factors such as noise, artifacts, and baseline wander. A higher SQI may indicate better signal quality and a lower SQI may indicate poorer signal quality. Similar to EHG, the SNR may indicate the ratio of the amplitude of the cardiac signal to the background noise. A higher SNR may signify a stronger ECG signal. A lower SNR may indicate a weaker ECG signal. In the lead voltage levels, different leads in an ECG recording may have varying signal strengths. Monitoring the voltage levels in each lead may indicate the overall signal strength.

At 420, a second signal measurement may be received. For example, the computing device 101, 205, 601 may receive the second signal measurement from a second sensor device 104b, 206. The second signal measurement may comprise one or more pressure changes (or one or more mechanical signals) measured at the second sensor device 104b, 206. The second sensor device 104b, 206 may be a pneumatic tocodynamometer (pTOCO) placed on the abdomen of a pregnant person to monitor the progress of labor and assess the frequency, duration, and intensity of contractions. The one or more pressure changes (or one or more mechanical signals) may be measured during a period of time. For example, the second sensor device 104b, 206 may measure the one or more pressure changes (or one or more mechanical signals) during the same period that the first sensor device 104a, 204 measures the one or more electrical signals. The one or more pressure changes (or one or more mechanical signals) may comprise one or more waveforms generated by amplifying the one or more pressure changes (or one or more mechanical signals) measured at the second sensor device 104b, 206 based on at least one movement of one or more uterine muscles of the pregnant person. Similar to the first sensor device 104a, 206, the second sensor device 104b, 206 may send the one or more waveforms using a wired or wireless communication interface (e.g., Bluetooth, cellular, Wi-Fi, Zigbee, NFC, or any other short/long range communication interface) equipped with or communicably accessible by the second sensor device 104b, 206 to the computing device 101, 205, 601, the server 106, or another computing device. The computing device 101, 205, 601 or another computing device may receive the one or more wave forms using a wired or wireless communication interface (e.g., Bluetooth) equipped with or communicably accessible by the computing device 101, 205, 601.

The second signal measurement may indicate a second parameter. The second parameter may be determined based on the one or more pressure changes (or one or more mechanical signals). For example, the computing device 101, 205, 601 may determine, based on the received one or more pressure changes (or one or more mechanical signals), the second parameter that may be indicative of pressure strength or the one or more pressure changes (or one or more mechanical signals). The second parameter may provide valuable information about the amplitude and characteristics of the one or more pressure changes (or one or more mechanical signals.

At 430, a contractile event indicative of a uterine contraction in a pregnant person may be determined based on at least one of the first parameter and the second parameter. The contractile event may comprise a positive uterine contraction and/or a negative uterine contraction. The uterine contractions may refer to the coordinated muscular actions of the uterine wall of the pregnant person that serve to prepare the body for childbirth and to facilitate the delivery of the baby during labor. The term uterine contraction may be interchangeably used as labor contractions throughout this disclosure. For example, the computing device 101, 205, 601 or another computing device may compare the first parameter to a first threshold and the second parameter to a second threshold. For example, the first threshold and the second threshold may be different. For example, the first threshold may be indicative of a uterine contraction level. For example, the second threshold may be indicative of a pressure level. For example, if the first parameter satisfies (e.g., is greater than or greater than or equal to) the first threshold but the second parameter does not satisfy (e.g., is less than or less than or equal to) a second threshold, the computing device 101, 205, 601 may determine that the uterine contraction has occurred for the pregnant person (e.g., a positive uterine contraction). For example, if at least one electrical signal measured by at least one electrode 204a-e satisfies the first threshold, the computing device 101, 205, 601 may determine that the uterine contraction has occurred on the subject (i.e., positive uterine contraction).

For example, if the computing device 101, 205, 601 or another computing device determines the first parameter does not satisfy (e.g., is less than or less than or equal to) a first threshold but the second parameter satisfies (e.g., is greater than or greater than or equal to) a second threshold, the computing device 101 may determine the uterine contraction has occurred for the pregnant person (e.g., the positive uterine contraction). For example, if the computing device 101, 205, 601 or another computing device determines that all electrical signals measured by all electrodes 204a-e do not satisfy the first threshold but the second parameter satisfies the second threshold, the computing device 101 may determine that the uterine contraction has occurred for the pregnant person (i.e., positive uterine contraction).

For example, if the computing device 101, 205, 601 or another computing device determines the first parameter satisfies (e.g., is greater than or greater than or equal to) a first threshold and the second parameter satisfies (e.g., is greater than or greater than or equal to) a second threshold, the computing device 101 or another computing device may determine that the uterine contraction has occurred for the pregnant person (e.g., the positive uterine contraction). For example, if at least one electrical signal measured by at least one electrode 204a-e satisfies the first threshold and the second parameter satisfies the second threshold, the computing device 101, 205, 601 or another computing device may determine that the uterine contraction has occurred for the pregnant person (e.g., the positive uterine contraction). For example, if the computing device 101, 205, 601 or another computing device determines the first parameter does not satisfy (e.g., is less than or less than or equal to) the first threshold and the second parameter does not satisfy (e.g., is less than or less than or equal to) the second threshold, the computing device 101, 205, 601 or another computing device may determine that the uterine contraction has not occurred (e.g., a negative uterine contraction).

Once the contractile event is determined, at 440, the indication of the contractile event may be sent to other devices. For example, the computing device 101, 205, 601 may send the result of the determined contractile event to the electronic device 102 and/or the server 106. The computing device 101, 205, 601 may send it using a wireless communication interface or a wired communication interface to other devices such as the electronic device 102 and/or the server 106.

It is noted that the method 400 in FIG. 4 may be performed by any other devices such as the electronic device 102 and/or the server 106. In the example where the electronic device 102 performs the method 400 in FIG. 4, the electronic device 102 may receive the first signal measurement from the first sensor device 104a and the second signal measurement from the second sensor device 104b. In the example where the server 106 performs the method 400 in FIG. 4, the server 106 may receive the first signal measurement from the first sensor device 104a and the second signal measurement from the second sensor device 104b. Additionally or alternatively, the server 106 may receive the first signal measurement and the second signal measurement though the computing device 101 or the electronic device 102. In other words, the computing device 101 or the electronic device 102 may hand over the first signal measurement and the second signal measurement to the server 106 for further processing.

A hospital setting may have ample WiFi bandwidth. In this example, the data acquisition or monitoring systems can stream all the data in real time to other units. However, in ‘at home’ situation (e.g., telemedicine cases) may have limited bandwidth. In this example, the bandwidth required to transmit high resolution fetal ECG may not be sufficient. Thus, methods, apparatuses, and/or systems that are able to adapt to provide needed data in a low bandwidth environment are beneficial, especially, when the patient is located in a low bandwidth wireless/wired environment. For example, a web application may request a fixed duration recording, sufficient to see several heart beats, be sent. Upon analysis, the web application may transmit back the coefficients of a data driven beamformer. Such a beamformer may be implemented by a 32-bit processor in real time. The purpose of the beamformer may be to preprocess the raw recording to suppress all signals except the fetal ECG ‘R’ waves, thus allowing the data recorder to efficiently extract the fetal beat to beat interval (e.g., heart rate). This may reduce the needed bandwidth dramatically. The telemedicine providers may observe the uterine activity and fetal heart rate in real time, and may review short recordings of the raw abdominal recordings. The methods/apparatuses/systems described herein may give an indication to the mother of fetal heartbeat via a blinking LED.

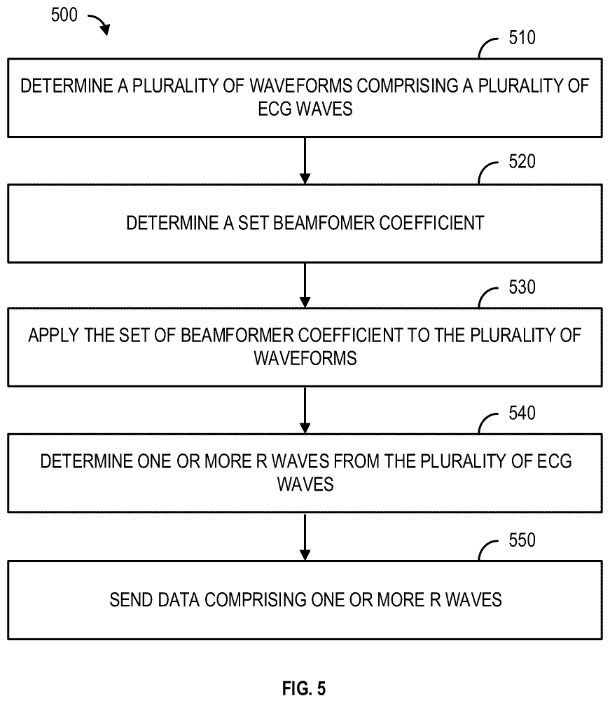

FIG. 5 shows an example method 500 for determining an adaptive bandwidth, which may be used in combination with any of other embodiments described herein. At 510, a plurality of waveforms that comprises a plurality of electrocardiogram (ECG) waves measured during a cardiac cycle may be determined based on a plurality of electrical signals received from the first sensor device 104a, 204. For example, the computing device 101, 205, 601 may determine a plurality of waveforms that comprises a plurality of ECG waves measured during a cardiac cycle. As described above, the first sensor device 104a, 204 may monitor fetal ECG or fetal heartrate based on the at least one electrodes 204a-e placed on the abdomen of a pregnant person. The first sensor device 104a, 204 may detect the electrical signals generated by the fetal heart. Specifically, the first sensor device 104a, 204 may record the electrical impulses generated by the fetal heart as it contracts and relaxes, producing a trace known as the fetal heart rate (FHR) graph. Fetal ECG is a technique used to monitor and record the electrical activity of the fetal heart during pregnancy.

The plurality of ECG waves may comprise a P wave, a Q wave, an R wave, an S wave, and a T wave. ECG waves may be the graphical representations of the electrical activity of the heart recorded by the first sensor device 104a, 204. The ECG waves and intervals observed in an ECG may represent different phases of the cardiac cycle. The plurality of ECG waves may comprise a P wave, a Q wave, an R wave, an S wave, and a T wave. The P wave may be the first wave in the ECG and represent the depolarization (i.e., contraction) of the atria, the upper chambers of the heart. The P wave may indicate the electrical impulse spreading through the atria and initiating the contraction. The QRS complex may be a composite of three waves, Q wave, R wave, and S waves, that follow the P wave. The QRS complex may represent the depolarization of the ventricles, the lower chambers of the heart. The Q wave may be the initial negative deflection. The R wave may be the first positive deflection. The S wave may be the second negative deflection. The T wave may be the wave that follows the QRS complex. The T wave may represent the repolarization (i.e., recovery) of the ventricles as they prepare for the next heartbeat.

As describe above, the R wave may be a positive deflection that occurs in the QRS complex, which is the main wave complex in the ECG. The QRS complex represents the depolarization (e.g., contraction) of the ventricles, the lower chambers of the heart. During a normal cardiac cycle, the depolarization starts at the atria, causing the P wave on the ECG. Then, the electrical impulse spreads to the ventricles, leading to their contraction. The QRS complex may represent this ventricular depolarization. The R wave may be the first positive deflection in the QRS complex, and its appearance on the ECG may indicate that the electrical impulse is spreading through the ventricles. The R wave may be followed by a negative deflection called the S wave and sometimes preceded by a smaller initial negative deflection called the Q wave (hence the term QRS complex).

At 520, a set of beamformer coefficients that is applied to the plurality of waveforms to filter the plurality of ECG waves may be determined. For example, a computing device 101, 205, 601 may determine a set of beamformer coefficients that is applied to the plurality of waveforms to filter the plurality of ECG waves. Alternatively or additionally, the computing device 101, 205, 601 may receive the set of beamformer coefficients from a server 106, an electronic device 102, or the like. The set of beamformer coefficients may comprise one or more complex values assigned to the plurality of waveforms to filter the plurality of ECG waves. The set of beamformer coefficients may refer to a set of weights or complex values assigned to each element of an array or sensor to optimize the formation of a directional beam or to extract specific signals from a set of received data in the context of data-driven beamforming. The set of beamformer coefficients may be used to adjust the weights or coefficients associated with each element of the sensor array based on the characteristics of the received data. These coefficients can be computed using various algorithms, such as the Minimum Variance Distortionless Response (MVDR), Capon, or Multiple Signal Classification (MUSIC) methods, among others. By optimizing the beamformer coefficients, the beamformer can shape the spatial response of the array, directing its sensitivity towards the direction of interest while minimizing sensitivity to other directions. This may result in improved signal-to-noise ratio and better spatial resolution.

At 530, one or more R waves from the plurality of ECG waves may be determined based on the filtering the plurality of ECG waves. For example, the computing device 101, 205, 601 may determine one or more R waves from the plurality of ECG waves based on filtering the plurality of ECG waves. Specifically, the set of beamformer coefficients may be applied to the plurality of waveforms to filter the plurality of ECG waves. In other words, the set of beamformer coefficients may be used to preprocess the raw recording of electrical signals and suppress all ECG waves except the R waves. This may allow the computing device 101, 205, 601 to efficiently extract the fetal heart rate and reduce the size of fetal ECG data to display. At 550, the data comprising the one or more R waves may be transmitted. For example, the computing device may generate the fetal ECG data with the filtered R waves, thereby reducing the size of fetal ECG data. Since the size of fetal ECG data is significantly reduced, the computing device 101 may enable the recipient such as the electronic device 102 or the sever 106 to receive the fetal ECG data (i.e., comprising R waves only) with a lower bandwidth than a bandwidth required to receive the unfiltered ECG data (i.e., comprising all ECG waves). It is noted that the method 500 in FIG. 5 may be performed by the electronic device 102 and/or the server 106.

Fetal breathing may be a sign of fetal wellbeing. With the methods/apparatuses/systems described herein, the bio-magnetic signal associated with fetal breathing may be recorded. It may be roughly the same amplitude as the fetal ECG which can be captured with abdominal electrodes. The fetal breathing in the abdominal recordings may be found based on the methods/apparatuses/systems described herein.

In an example, the behavior of uterine contractions may be analyzed based on quadrants of activity with the biomagnetic recording system described herein. The sensor or electrode (e.g., four electrodes) arrangement may allow the biomagnetic recording system to extract similar information. Normal contractions predominantly start in the upper left quadrant and proceed to one of the other quadrants. False labor and active labor may be identified based on the methods/apparatuses/systems described herein.

In an example, fetal orientation (e.g., breach or engaged) may be inferred by observing which channels the fetal ECG appears dominate and by observing the polarity of the fetal ‘R’ wave. In an embodiment, a computing device 101, an electronic device 102 or a server 106 may send a message to the patient, such as “replace electrode #3.”

In an example aspect, the methods, apparatuses, and systems can be implemented on a computer 601 as illustrated in FIG. 6 and described below. By way of example, the computing device 101 of FIG. 1 can be a computer 601 as illustrated in FIG. 6. Similarly, the methods, apparatuses, and systems disclosed can utilize one or more computers to perform one or more functions in one or more locations. For example, the computer 601 may perform or implement the methods or processes described in FIGS. 4-5 based on the communication with the sensor devices 104a-b. FIG. 6 is a block diagram illustrating an example operating environment 600 for performing the disclosed methods. This example operating environment 600 is only an example of an operating environment and is not intended to suggest any limitation as to the scope of use or functionality of operating environment architecture. Neither should the operating environment 600 be interpreted as having any dependency or requirement relating to any one or combination of components illustrated in the example operating environment 600.

The present methods, apparatuses, and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, and/or the like that perform particular tasks or implement particular abstract data types. The disclosed methods can also be practiced in grid-based and distributed computing environments where tasks are performed by remote processing devices that are linked through a communications network. In a distributed computing environment, program modules can be located in local and/or remote computer storage media such as memory storage devices.

Further, one skilled in the art will appreciate that the systems, apparatuses, and methods disclosed herein can be implemented via a general-purpose computing device in the form of a computer 601. The computer 601 can comprise one or more components, such as one or more processors 603, a system memory 610, and a bus 611 that couples various components of the computer 601 comprising the one or more processors 603 to the system memory 610. The system can utilize parallel computing.

The bus 611 can comprise one or more of several possible types of bus structures, such as a memory bus, memory controller, a peripheral bus, an accelerated graphics port, or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 611, and all buses specified in this description can also be implemented over a wired or wireless network connection and one or more of the components of the computer 601, such as the one or more processors 603, a mass storage device 604, an operating system 605, a network adapter 609, the system memory 610, an Input/Output Interface 607, a display adapter 606, a display device 612, and a human machine interface 602, can be contained within one or more remote computing devices 613a,b,c at physically separate locations, connected through buses of this form, in effect implementing a fully distributed system.

The computer 601 typically comprises a variety of computer readable media. Example readable media can be any available media that is accessible by the computer 601 and comprises, for example and not meant to be limiting, both volatile and non-volatile media, removable and non-removable media. The system memory 610 can comprise computer readable media in the form of volatile memory, such as random access memory (RAM), and/or non-volatile memory, such as read only memory (ROM). The system memory 610 typically can comprise data such as the operating system 605 that is accessible to and/or is operated on by the one or more processors 603.

In an embodiment, the computer 601 can also comprise other removable/non-removable, volatile/non-volatile computer storage media. The mass storage device 604 can provide non-volatile storage of computer code, computer readable instructions, data structures, program modules, and other data for the computer 601. For example, the mass storage device 604 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 604, such as, by way of example, the operating system 605. The operating system 605 can comprise elements of the programming and be stored on the mass storage device 604. Examples of such databases comprise, DB2®, Microsoft® Access, Microsoft® SQL Server, Oracle®, mySQL, PostgreSQL, and the like. The databases can be centralized or distributed across multiple locations within the network 614.

In an embodiment, the user can enter commands and information into the computer 601 via an input device (not shown). Examples of such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices can be connected to the one or more processors 603 via the human machine interface 602 that is coupled to the bus 611, but can be connected by other interface and bus structures, such as a parallel port, game port, an IEEE 1394 Port (also known as a Firewire port), a serial port, a network adapter 609, and/or a universal serial bus (USB).

In an embodiment, the display device 612 can also be connected to the bus 611 via an interface, such as the display adapter 606. It is contemplated that the computer 601 can have more than one display adapter 606 and the computer 601 can have more than one display device 612. For example, the display device 612 can be a monitor, an LCD (Liquid Crystal Display), light emitting diode (LED) display, television, smart lens, smart glass, and/or a projector. In addition to the display device 612, other output peripheral devices can comprise components such as speakers (not shown) and a printer (not shown) which can be connected to the computer 601 via an Input/Output Interface 607. Any step and/or result of the methods can be output in any form to an output device. Such output can be any form of visual representation, comprising, but not limited to, textual, graphical, animation, audio, tactile, and the like. The display device 612 and the computer 601 can be part of one device, or separate devices.

The computer 601 can operate in a networked environment using logical connections to one or more remote computing devices 613a,b,c. By way of example, a remote computing device 613a,b,c can be a personal computer, computing station (e.g., workstation), portable computer (e.g., laptop, mobile phone, tablet device), smart device (e.g., smartphone, smart watch, activity tracker, smart apparel, smart accessory), security and/or sensor device, a server, a router, a network computer, a peer device, edge device or other common network node, and so on. Logical connections between the computer 601 and a remote computing device 613a,b,c can be made via a network 614, such as a local area network (LAN) and/or a general wide area network (WAN). Such network connections can be through the network adapter 609. The network adapter 609 can be implemented in both wired and wireless environments. Such networking environments are conventional and commonplace in dwellings, offices, enterprise-wide computer networks, intranets, and the Internet.

For purposes of illustration, application programs and other executable program components such as the operating system 605 are illustrated herein as discrete blocks, although it is recognized that such programs and components can reside at various times in different storage components of the computing device 601, and are executed by the one or more processors 603 of the computer 601. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Example computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

For purposes of illustration, application programs and other executable program components are illustrated herein as discrete blocks, although it is recognized that such programs and components can reside at various times in different storage components. An implementation of the described methods can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Example computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as example only, with a true scope and spirit being indicated by the following claims.

What is claimed is:

1. A method comprising:
receiving, from a first sensor device, a first signal measurement indicative of a first parameter;
receiving, from a second sensor device, a second signal measurement indicative of a second parameter; and
determining, based on at least one of the first parameter or the second parameter, a contractile event indicative of a uterine contraction.

2. The method of claim 1, wherein the first sensor device comprises at least one electrode and wherein the first signal measurement comprises one or more electrical signals.

3. The method of claim 1, wherein the second sensor device is a pneumatic tocodynamometer (pTOCO).

4. The method of claim 1, wherein the second signal measurement comprises one or more pressure changes measured at the second sensor device.

5. The method of claim 1, wherein determining the contractile event indicative of the uterine contraction comprises:
determining the first parameter satisfies a first threshold;
determining the second parameter does not satisfy a second threshold; and
determining, based the first parameter satisfying the first threshold and the second parameter not satisfying the second threshold, the contractile event.

6. The method of claim 1, wherein determining the contractile event indicative of the uterine contraction comprises:
determining the first parameter does not satisfy a first threshold;
determining the second parameter satisfies a second threshold; and
determining, based on the first parameter not satisfying the first threshold and based on the second parameter satisfying the second threshold, the contractile event.

7. The method of claim 1, wherein determining the contractile event indicative of the uterine contraction comprises:
determining the first parameter satisfies a first threshold;
determining the second parameter satisfies a second threshold; and
determining, based on the first parameter satisfying the first threshold and based on the second parameter satisfying the second threshold, the contractile event.

8. The method of claim 1, further comprising:
receiving, from the first sensor device, a third signal measurement indicative of a third parameter;
receiving, from the second sensor device, a fourth signal measurement indicative of a fourth parameter;
determining the third parameter does not satisfy a first threshold;
determining the fourth parameter does not satisfy a second threshold; and
determining, based on the third parameter not satisfying the first threshold and based on the fourth parameter not satisfying the second threshold, a second uterine contraction did not occur.

9. The method of claim 1, wherein the first signal measurement and the second signal measurement are measured during a period of time.

10. A system comprising:
a first sensor device configured to perform a first signal measurement indicative of a first parameter;
a second sensor device configured to perform a second signal measurement indicative of a second parameter; and
a computing device configured to determine, based on at least one of the first parameter or the second parameter, a contractile event indicative of a uterine contraction.

11. The system of claim 10, wherein the first sensor device comprises at least one electrode and wherein the first signal measurement comprises one or more electrical signals.

12. The system of claim 10, wherein the second sensor device is a pneumatic tocodynamometer (pTOCO) and wherein the second signal measurement comprises one or more pressure changes measured at the second sensor device.

13. The system of claim 10, wherein the computing device is further configured to:
determine the first parameter satisfies a first threshold;
determine the second parameter satisfies a second threshold; and
determine, based on the first parameter satisfying the first threshold and the second parameter satisfying the second threshold, the contractile event.

14. The system of claim 10, wherein the computing device is further configured to send at least one of the contractile event indicative of the uterine contraction, the first signal measurement, or the second signal measurement.

15. An apparatus comprising:
a first belt portion;
a first sensor device disposed along the first belt portion;
a second belt portion;
a second sensor device disposed along the second belt portion; and
a hinge coupling the first belt portion to the second belt portion.

16. The apparatus of claim 15, further comprising a computing device disposed along one of the first belt portion or the second belt portion.

17. The apparatus of claim 15, wherein the first belt portion comprises a first belt and the second belt portion comprises a second belt.

18. The apparatus of claim 15, wherein the second belt portion is configured to rotate about an axis with respect to the first belt portion by way of the hinge coupling.

19. The apparatus of claim 15, further comprising a transmitter communicably coupled to the first sensor device and the second sensor device.

20. The apparatus of claim 15, wherein the second sensor device comprises a pneumatic tocodynamometer.
